(19) United States
(12) Patent Application Publication
(10) Pub. No.: US 2025/0149175 A1
(43) Pub. Date: May 8, 2025

(54) DEVICE AND METHOD FOR DIAGNOSIS OF DYSGRAPHIA

(71) Applicant: QATAR UNIVERSITY, Doha (QA)

(72) Inventors: Jayakanth KUNHOTH, Doha (QA); Somaya AL MAADEED, Doha (QA); Moutaz SALEH, Doha (QA); Younes AKBARI, Doha (QA)

(21) Appl. No.: 18/942,300

(22) Filed: Nov. 8, 2024

Related U.S. Application Data
(60) Provisional application No. 63/547,716, filed on Nov. 8, 2023.

(51) Int. Cl. G16H 50/20; G06N 20/00

(52) U.S. Cl. CPC G16H 50/20; G06N 20/00

(57) ABSTRACT

Systems, methods, apparatuses, and computer program products for the diagnosis of dysgraphia and a method of using the same. A method may include identifying one or more reference machine learning (ML) models that that are associated with dysgraphia. The method may also include capturing input from a user via an input device connected to a force sensor resistor (FSR). The method may further include collecting data from the FSR and one or more sensors in contact with the user. The method may also include analyzing the collected data and the captured input against the one or more reference ML models. Additionally, the method may include determining based on inference results generated from the one or more reference ML models whether the input is indicative of dysgraphia. Further, the method may include updating the one or more reference ML models with the captured input and collected data.

### CROSS REFERENCE TO RELATED APPLICATION

This application claims the priority of U.S. Provisional Patent Application No. 63/547,716, filed Nov. 8, 2023, which is hereby incorporated herein by reference in its entirety.

### FIELD

The present disclosure relates to diagnostic tools. More particularly the present disclosure relates to a system for the diagnosis of dysgraphia and a method of using the same.

### BACKGROUND

Dysgraphia is a writing disorder that primarily affects individuals' writing skills, particularly observed in children and elderly people with neurological conditions. The disability can have adverse effects on academic performance, hinder skill development, reduce confidence, and increase social anxiety. Unfortunately, dysgraphia is often misconceived as a lack of intelligence, leading to inadequate treatment and support. Early diagnosis and intervention are crucial for effective treatment. Current approaches to diagnosing dysgraphia, however, are heavily reliant on human resources and are both labor-intensive and time consuming.

Team-based assessments are one known way for diagnosing. The team-based assessments include multiple specialists from different domains such as education (teacher), psychology (occupational therapist), medicine (speech therapists, ophthalmologists), etc. These specialists jointly analyze the student's handwriting ability as well as other factors which can affect handwriting. It may be important to contemplate various contributing factors of dysgraphia such as speed and legibility of writing, inconsistency between spelling, ability, and verbal intelligence quotient, as well as how the pencil grip is held while writing and writing pose to effectively assess the condition of the student. Additionally, there is a lack of common medical assessment methods for examining the existence of dysgraphia.

Another process involves manual assessments conducted by experts from various domains, such as educators, occupational therapists, and speech-language pathologists. These professionals need to coordinate their schedules, conduct thorough evaluations, and rely on subjective observations and feedback from the child and parents. However, this approach is susceptible to human bias, leading to inconsistent interpretations and potential misdiagnoses. Additionally, the reliance on expert availability and the intricate coordination of different professionals can delay interventions and support

Tablet-based techniques are a popular approach for dysgraphia diagnosis. Tablet-based techniques can explore more characteristics of handwriting, which have turned out to be significant for the detection of dysgraphia. The data acquisition in this approach involves writing with a conventional pen or an electronic pen on paper overlaid on a tablet. However, the lower friction surface of tablet computers modifies graphomotor execution, which in turn contradicts the purpose. The altered graphomotor execution caused by the tablet's lower friction surface contradicts the natural writing process, potentially affecting the validity of captured data. Moreover, the pressure sensitivity of these tablets may vary depending on the model. Even though the grip force between the hands and writing instrument has a substantial correlation to the fine motor performance and the improper handling of the pen is prevalent in motor dysgraphia, it is not considered as an attributing factor for dysgraphia prediction. In addition, a single task (e.g., copying/writing/drawing) is not sufficient for diagnosing dysgraphia.

The above discussed limitations associated with known techniques collectively underscore the need for a more precise and consistent approach in dysgraphia diagnosis.

### SUMMARY

Some embodiments may be directed to a computer implemented method for diagnosing dysgraphia. The method may include identifying one or more reference machine learning (ML) models that that are associated with dysgraphia. The method may also include capturing input from a user via an input device connected to a force sensor resistor (FSR). The method may further include collecting data from the FSR and one or more sensors in contact with the user. In addition, the method may include analyzing the collected data and the capture user input against the one or more reference ML models. Further, the method may include determining based on inference results generated from the one or more reference ML models whether the user input is indicative of dysgraphia. The method may also include updating the one or more reference ML models with the captured input and collected data.

Other embodiments may be directed to a device for diagnosing dysgraphia. The device may include at least one processor, and at least one memory storing instructions, wherein the instructions, when executed by the at least one processor, cause the apparatus at least to identify one or more reference machine learning (ML) models that that are associated with dysgraphia. The apparatus may also be caused to capture input from a user from an input device connected to force sensor resistor (FSR). The apparatus may further be caused to collect data from the FSR and one or more sensors in contact with the user. In addition, the apparatus may be caused to analyze the collected data and the captured input against the one or more reference ML models. Further, the apparatus may be caused to determine based on inference results generated from the one or more reference ML models whether the input is indicative of dysgraphia. The apparatus may also be caused to update the one or more ML models with the captured input and collected data.

Other embodiments may be directed to an apparatus. The apparatus may include means for identifying one or more reference machine learning (ML) models that that are associated with dysgraphia. The apparatus may also include means for capturing input from a user via an input device connected to a force sensor resistor (FSR). The apparatus may further include means for collecting data from the FSR and one or more sensors in contact with the user. In addition, the apparatus may include means for analyzing the collected data and the capture user input against the one or more reference ML models. The apparatus may also include means for determining based on inference results generated from the one or more reference ML models whether the user input is indicative of dysgraphia. The apparatus may further include means for updating the one or more reference ML models with the captured input and collected data.

Other example embodiments may be directed to a computer program product that performs a method. The method may include identifying one or more reference machine learning (ML) models that that are associated with dysgraphia. The method may also include capturing input from a user via an input device connected to a force sensor resistor (FSR). The method may further include collecting data from the FSR and one or more sensors in contact with the user. In addition, the method may include analyzing the collected data and the capture user input against the one or more reference ML models. Further, the method may include determining based on inference results generated from the one or more reference ML models whether the user input is indicative of dysgraphia. The method may also include updating the one or more reference ML models with the captured input and collected data.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detail description serve to explain the principles of the invention. In the drawings:

FIG. 1 illustrates an example system and framework, according to certain embodiments, according to certain embodiments.

FIG. 2A illustrates a perspective view of an exemplary embodiment of a writing utensil that makes up part of the system of FIG. 1, according to certain embodiments.

FIG. 2B illustrates a plan view of the writing utensil of FIG. 2A, according to certain embodiments.

FIG. 2C illustrates another perspective view of the writing utensil of FIG. 2A, according to certain embodiments.

FIG. 2D illustrates yet another perspective view of the writing utensil of FIG. 2A, according to certain embodiments.

FIG. 3A illustrates a perspective view of an exemplary embodiment of a electromyography band that makes up part of the system of FIG. 1, according to certain embodiments.

FIG. 3B illustrates a side view of the electromyography band of FIG. 3A, according to certain embodiments.

FIG. 3C illustrates a plan view of the electromyography band of FIG. 3A, according to certain embodiments.

FIG. 4 illustrates a block diagram of the system of FIG. 1, according to certain embodiments.

FIG. 5 illustrates an exemplary workflow for a decision making process for system of FIG. 1, according to certain embodiments.

FIG. 6A illustrates readings from the system of FIG. 1 when a patient writes various letters, according to certain embodiments.

FIG. 6B illustrates additional readings from the system of FIG. 1 when a patient writes various letters, according to certain embodiments.

FIG. 7A illustrates readings from the system of FIG. 1 when a patient writes a word, according to certain embodiments.

FIG. 7B illustrates additional readings from the system of FIG. 1 when a patient writes a word, according to certain embodiments.

FIG. 8A illustrates readings from the system of FIG. 1 when a patient draws a shape, according to certain embodiments.

FIG. 8B illustrates additional readings from the system of FIG. 1 when a patient draws a shape, according to certain embodiments.

FIG. 9 illustrates an example flow diagram of a method, according to certain embodiments.

FIG. 10 illustrates an example apparatus, according to certain embodiments.

### DETAILED DESCRIPTION

It will be readily understood that the components of certain example embodiments, as generally described and illustrated in the figures herein, may be arranged and designed in a wide variety of different configurations. The following is a detailed description of some embodiments for assisting people with visual impairments.

The features, structures, or characteristics of example embodiments described throughout this specification may be combined in any suitable manner in one or more example embodiments. For example, the usage of the phrases “certain embodiments,” “an example embodiment,” “some embodiments,” or other similar language, throughout this specification refers to the fact that a particular feature, structure, or characteristic described in connection with an embodiment may be included in at least one embodiment. Thus, appearances of the phrases “in certain example embodiments,” “an example embodiment,” “in some example embodiments,” “in other example embodiments,” or other similar language, throughout this specification do not necessarily all refer to the same group of embodiments, and the described features, structures, or characteristics may be combined in any suitable manner in one or more example embodiments.

Additionally, if desired, the different functions or steps discussed below may be performed in a different order and/or concurrently with each other. Furthermore, if desired, one or more of the described functions or steps may be optional or may be combined. As such, the following description should be considered as merely illustrative of the principles and teachings of certain example embodiments, and not in limitation thereof.

Learning disabilities is distinguishable from lack of intelligence. For example, learning disabilities encompass a broad category of learning disorders that impede the acquisition of skills in individuals. Research indicates that individuals with learning disabilities possess intelligence levels that are at least average. While learning disabilities may not significantly impact innate intelligence, they can lead to a lack of self-esteem and self-confidence, particularly in children. Children naturally strive to learn new information and acquire skills as they grow. However, learning disabilities may hinder or delay this skill acquisition, causing children to struggle with grasping new concepts and feeling inferior to their peers. Consequently, reduced self-esteem, confidence, and various socio-emotional and behavioral challenges can arise. Learning disabilities impair a student's perception abilities, resulting in difficulties in various tasks such as reading, writing, and mathematics. Dysgraphia, a specific type of learning disability, primarily manifests as difficulties in written expression. Dysgraphia not only affects handwriting but also impacts grammar, spelling, and organizational skills. Available statistics on learning disabilities indicate that approximately 10-30% of children worldwide encounter challenges specifically related to handwriting.

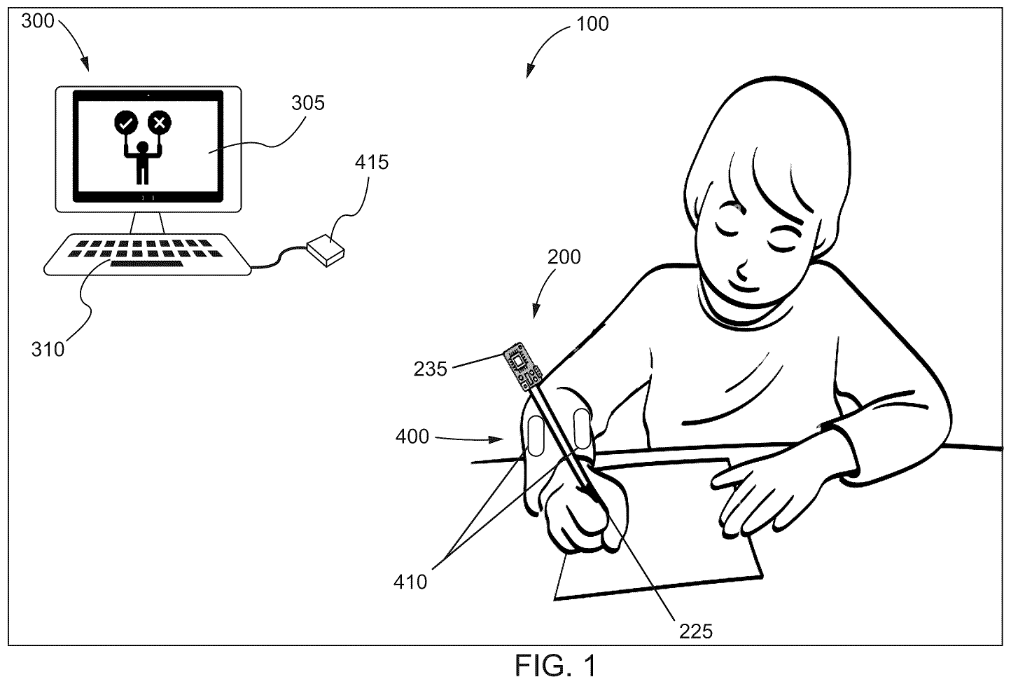

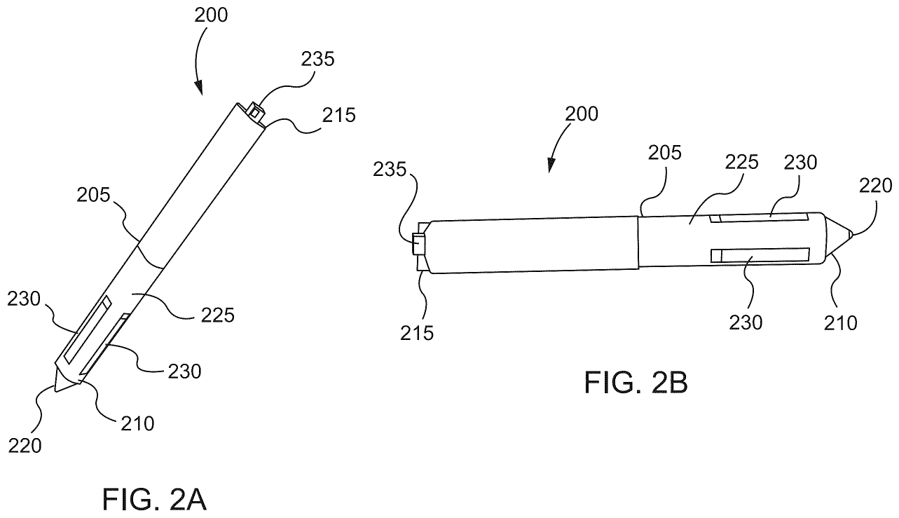

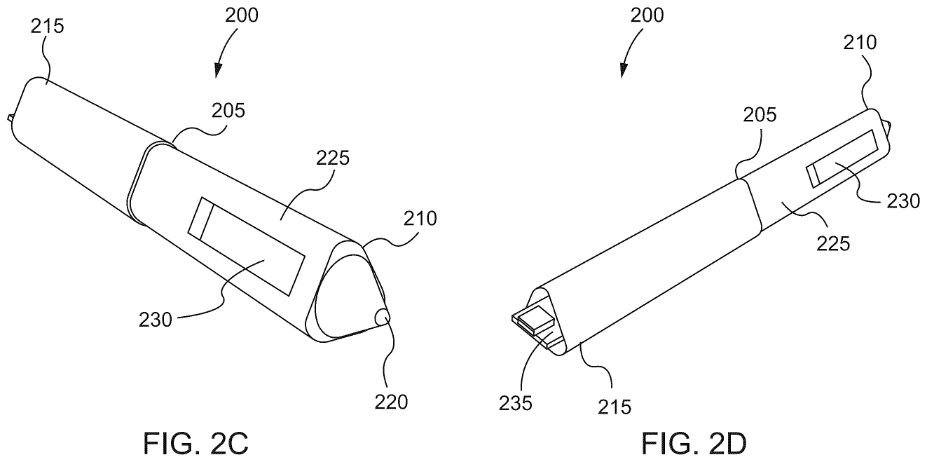

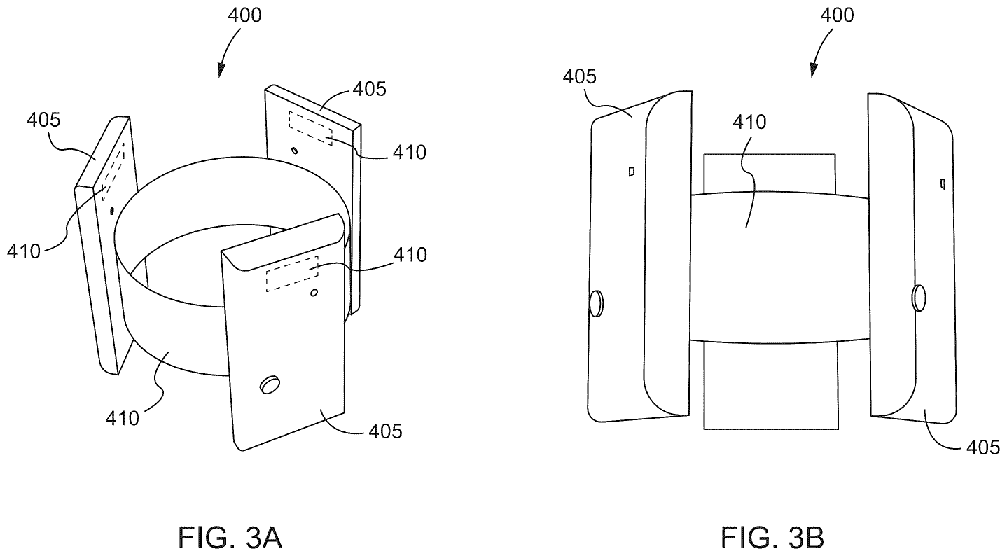

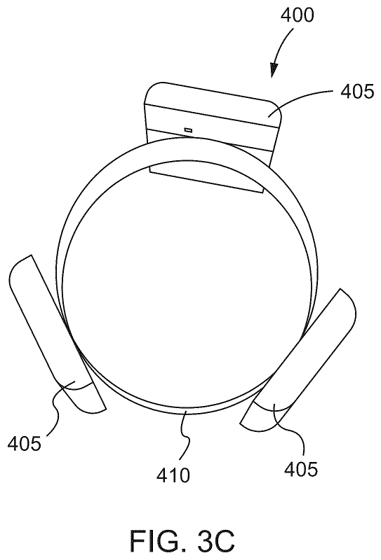

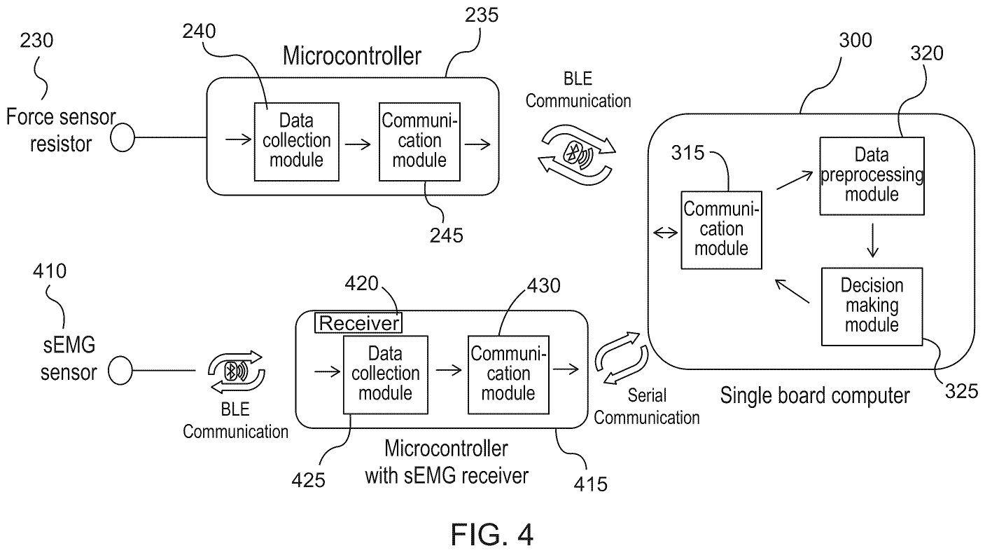

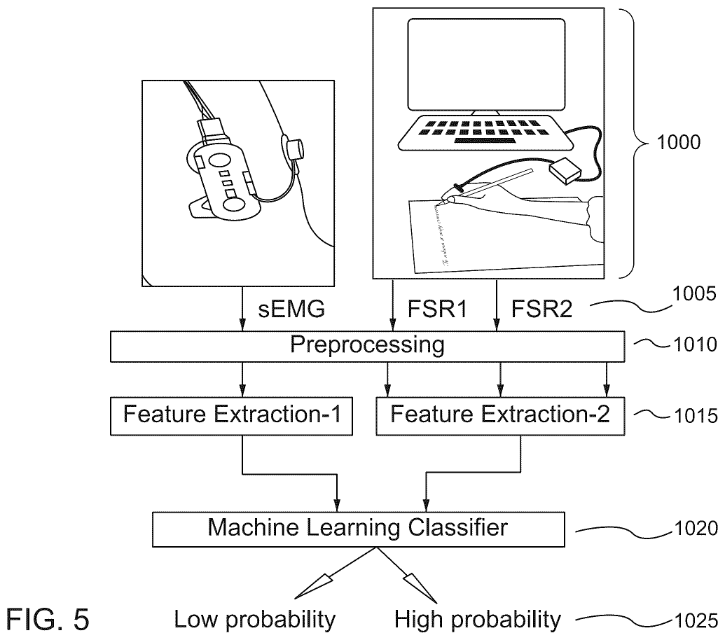

FIGS. 1-5 show an exemplary embodiment of a system for the diagnosis of dysgraphia. The system 100 includes a writing utensil 200, a computer 300, and an electromyograph band 400. According to one example embodiment, the writing utensil 200 is a pen. In alternative embodiments, the writing utensil 200 may be any desired device (e.g., pencil, stylus). The electromyograph band 400 is configured for attachment to the forearm of a patient as the patient writes.

The computer 300 is configured to receive and process information from the writing utensil 200 and electromyograph band 400, as will be discussed further below. According to one example embodiment, the computer 300 is a standalone single-board computer with an Arduino Uno microcontroller, a screen 305, a keyboard 310, and runs Linux software. In alternative embodiments, the computer may be any desired device.

The writing utensil 200 includes a body 205 that extends between a first end 210 and a second end 215. A marking device 220 (e.g., graphite dispenser, ink dispenser) is provided at the first end 210 of the body 205. A grip is provided adjacent the marking device 220. The grip 225 includes a force sensor 230. According to one example embodiment, the force sensor 230 includes two force sensor resistors (“FSR”). In alternative embodiments, the writing utensil 200 may include a fewer or greater number of force sensor 230 resistors. In other alternative embodiments, the force sensor 230 may be any desired arrangement (e.g., load cell).

A first microcontroller 235 is provided at a second end 215 of the body 205. According to one example embodiment, the first microcontroller 235 is an Arduino Nano BLE 33 sense nano microcontroller. In alternative embodiments, the first microcontroller may be any desired arrangement.

The first microcontroller 235 is in communication with the computer 300 and is configured to collect readings from the force sensor 230. The first microcontroller 235 may include a data collection module 240 and a communication module 245. The data collection module 240 is configured to extract sensor measurements from the force sensor 230. The communication module 245 is configured to transfer the sensor measurements to the computer 300. According to one example embodiment, the system 100 uses Bluetooth low energy protocol to transfer data from the first microcontroller 235 to the computer 300. In alternative embodiments, any desired arrangement may be used.

The electromyograph band 400 includes paddles 405 that are mounted on an elastic band 410. A surface electromyography (“sEMG”) sensor 410 is mounted to each one of the paddles 405. According to one example embodiment, the electromyograph band 400 may be and 8-channel wireless band capable of capturing signals at 500 Hz. In alternative embodiments, the electromyograph band may include a fewer or greater number of paddles. In other alternative embodiments, the paddles may be omitted and the surface electromyography sensors may be mounted directly on the elastic band. In still other alternative embodiments, the elastic band may be omitted and the surface electromyography sensors may include an arrangement for directly mounting the surface electromyography sensors to a patient (e.g., temporary adhesive).

A second microcontroller 415 is associated with electromyography band 400. The second microcontroller 415 includes a surface electromyography receiver 420 that is in communication with the computer 300. The surface electromyography receiver 420 is configured to collect readings from the surface electromyography sensors 410 of the electromyography band 400. According to one example embodiment, the surface electromyography receiver 420 collects readings from the surface electromyography sensors 410 wirelessly. For example, the surface electromyography receiver 420 may use Bluetooth low energy protocol. In alternative embodiments, any desired arrangement may be used.

The computer 300 is equipped with data analysis software that is configured to analyze the readings from the writing utensil and the electromyography band in order to detect the existence of dysgraphia. In addition to analysis, the computer may be used for data storage and configured to receive software support to keep the computer up to date.

The computer 300 further includes a communication module 315, a data pre-processing module 320, and a decision-making module 325. The communication module 315 is configured to maintain the communication between the first and second microcontrollers 235, 415 and to receive readings from the from the writing utensil 200 and the electromyography band 400. Once readings are received, they may be forwarded to the data pre-processing module 320. The data pre-processing module is configured to convert the readings into meaningful full features, which are explained in further detail in Table 1 and Table 2 below.

TABLE 1
Features from FSR Values

| Feature | Definition | Equation |
|---|---|---|
| Mean | Average value of the FSR readings | $Mean = \frac{1}{N} {\sum }_{i = 1}^{N} F S {R}_{i}$ |
| Maximum | Maximum value of the FSR | Maximum = max(FSR₁, FSR₂, . . . , FSR_{N}) |
| | readings | |
| Minimum | Minimum value of the FSR | Minimum = min(FSR₁, FSR₂, . . . , FSR_{N}) |
| | readings | |
| Standard Deviation | Measure of variability in the FSR values | $Standard Deviation = \sqrt{\frac{1}{N} {\sum }_{i = 1}^{N} {( {FSR}_{i} - Mean )}^{2}}$ |
| Range | Difference between the | Range = Maximum − Minimum |
| | maximum and minimum | |
| | values | |
| Median | Middle value in the sorted | N/A (obtained directly from sorted FSR values) |
| | FSR readings | |
| Skewness | Measure of the asymmetry of the FSR data distribution | $Skewness = \frac{1}{N} {\sum }_{i = 1}^{N} {( \frac{{FS R}_{i} - Mean}{Standard Deviation} )}^{3}$ |
| Kurtosis | Measure of the peakedness of the FSR data distribution | $Kurtosis = \frac{1}{N} {\sum }_{i = 1}^{N} {( \frac{{FS R}_{i} - Mean}{Standard Deviation} )}^{4} - 3$ |
| Energy | Measure of the overall intensity or magnitude of the FSR values | $Energy = \sqrt{\frac{1}{N} {\sum }_{i = 1}^{N} F S {R}_{i}^{2}}$ |
| Time | Rate of change or | N/A (specific equations depend on chosen |
| Derivatives | acceleration of the FSR | differentiation technique) |
| | values | |

TABLE 2
Features from EMG Values

| Feature | | | |
|---|---|---|---|
| Type | Feature | Definition | Equation |
| Time domain | Mean absolute value | Average absolute amplitude of | $M A V = \frac{1}{N} {\sum }_{i = 1}^{N} \vert {x}_{i} \vert$ |
| | | the signal. | |
| | Root mean square | Square root of the average of squared | $RMS = \sqrt{\frac{1}{N} {{\sum }_{i = 1}}^{N} {x}_{i}^{2}}$ |
| | | amplitudes | |
| | Zero crossing rate | Rate of sign changes in the signal | $Z C R = \frac{1}{2 N} {\sum }_{i = 1}^{N - 1} \vert sign ( {x}_{i} ) - sign ( {x}_{i + 1} ) \vert$ |
| | Variance | Measure of signal dispersion | $Variance = \frac{1}{N} {\sum }_{i = 1}^{N} {( {x}_{i} - Mean )}^{2}$ |
| | | around the | |
| | | mean | |
| | Waveform length | Cumulative sum of absolute differences | $W L = {\sum }_{i = 1}^{N - 1} \vert {x}_{i + 1} - {x}_{i} \vert$ |
| | | between | |
| | | consecutive | |
| | | samples | |
| Frequency domain | Mean | Average frequency content of the signal | $M N F = \frac{{\sum }_{i = 1}^{N} {f}_{i} \cdot {P}_{i}}{{\sum }_{i = 1}^{N} {P}_{i}}$ |
| | Power | Distribution | PSD = FFT(x) · FFT(x)* |
| | Spectral | of signal power | |
| | Density | across | |
| | | frequencies | |
| Wave form | Number of | Total count | N/A (peak detection algorithm used) |
| morphology | peaks | of peaks in the | |
| | | signal | |
| | Rise Time | Time duration | N/A (based on peak detection algorithm) |
| | | between a | |
| | | certain | |
| | | threshold | |
| | | level and the | |
| | | peak amplitude | |
| Statistical | Skewness | Measure of asymmetry in the signal | $Skewness = \frac{1}{N} {\sum }_{i = 1}^{N} {( \frac{{x}_{i} - Mean}{Standard Deviation} )}^{3}$ |
| | | distribution. | |
| | Kurtosis | Measure of the peakedness of the signal | $Kurtosis = \frac{1}{N} {\sum }_{i = 1}^{{N}_{}} {( \frac{{x}_{i} - Mean}{Standard Deviation} )}^{4} - 3$ |
| | | distribution. | |
| | Standard deviation | Measure of signal dispersion around the mean. | $Standard Deviation = \sqrt{\frac{1}{N} {\sum }_{i = 1}^{N} {( {x}_{i} - Mean )}^{2}}$ |

The statistical values (mean, median, mode, maximum, minimum, 95th, and 5th percentile) of grip force, which are values obtained from the force sensor 230 of the writing utensil 200, and statistical values as well as root mean square and mean absolute value of sEMG signals, which are values obtained from the surface electromyography sensors 410 of the electromyography band 400, may be computed to form a feature vector.

This feature vector is forwarded to the decision-making module for classification into either a positive class (dysgraphia) or a negative (i.e., non-dysgraphia) class. The decision-making module 325 may be equipped with a machine learning model trained with data collected from a large population of people with dysgraphia writing and also people without dysgraphia writing. For instance, in some example embodiments, a person may write using the writing utensil, the FSR may measure grip pressure, and the sEMG sensor band may record muscle activation in the arm/biceps. The Arduino Nano may collect and transmit grip pressure data via BLE, and the WiFi receiver may forward EMG signals to the computer. The single-bard computer may analyze the received data using the machine learning model, and the system may classify the writing as normal or indicative of dysgraphia. According to certain example embodiments, the machine learning model may be updated/trained with new data received from the person/patient using the writing utensil and sEMG band, and utilize the new and old data to determine whether the writing/drawing by the patient/person is indicative of one with dysgraphia. In other example embodiments, the machine learning model may be adaptable to different groups of people (e.g., by age) and conditions/environmental settings (e.g., school, work, healthcare facility, office, home, etc.). As such, with the system of certain embodiments, it may be possible to reduce reliance on subject observations, while increasing consistency and reliability in diagnosis. It may also be possible to provide a more comprehensive analysis of the collected data compared to single-metric systems.

The accuracy of a diagnosis is dependent on the quality and diversity of the training dataset used to train the machine learning model. If the dataset is inadequate or lacks sufficient dysgraphia cases, the accuracy of the diagnosis may be compromised. Training dataset can be enhanced by collecting data from a diverse population, including individuals with various writing styles, physical conditions, and cultural backgrounds. This may improve the generalizability of the machine learning model and increase its accuracy in diagnosing dysgraphia. Furthermore, bias in the machine learning model is a major concern. To avoid bias, the machine learning model may be trained with data collected from subjects with different dialects.

The system of certain embodiments may implement a structured data collection process that involves a wide range of participants, including people with and without dysgraphia. For instance, certain embodiments may incorporate various writing tasks and scenarios to capture the full spectrum of writing styles and motor behaviors. This diverse dataset may allow the algorithm of certain embodiments to learn and adapt to the unique patterns associated with dysgraphia.

The system of certain embodiments may collect personal and sensitive data, such as muscle activation patterns. Proper data handling and privacy measures may be implemented to ensure the security and confidentiality of the user's information.

In use, a patient may use the writing utensil 200 to write certain sentences and words while the electromyography band is attached to the forearm of the patient. Advantageously, the patient can write on a pad of paper, thereby preventing any alteration of the graphomotor execution. The force exerted on the writing utensil 200 by the patient while writing is measured by the force sensor 230. The muscle movement activations are sensed by the surface electromyography sensors 410 of the electromyography band 400. Thus, the system 100 allows for real-time measurement of the pressure exerted by the patient's hand on the writing utensil 200 while simultaneously tracking the patient's muscle movements.

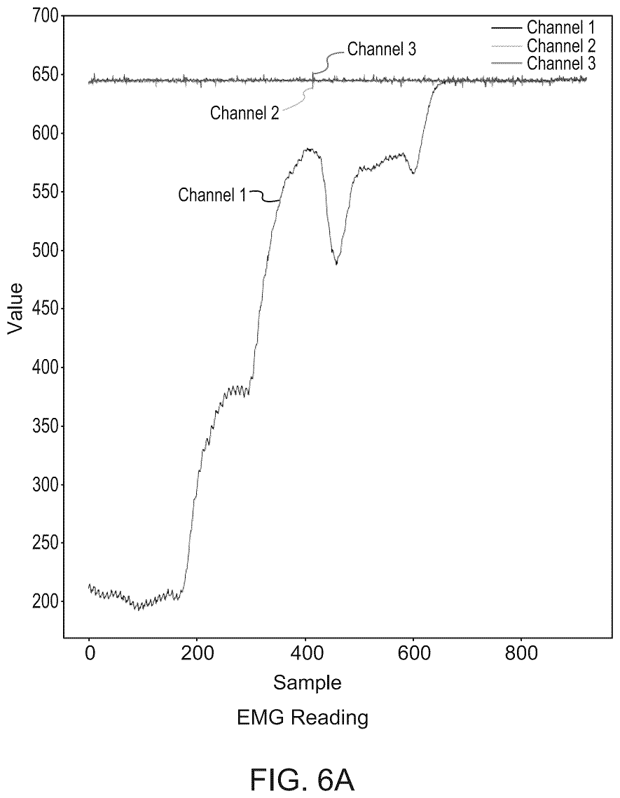

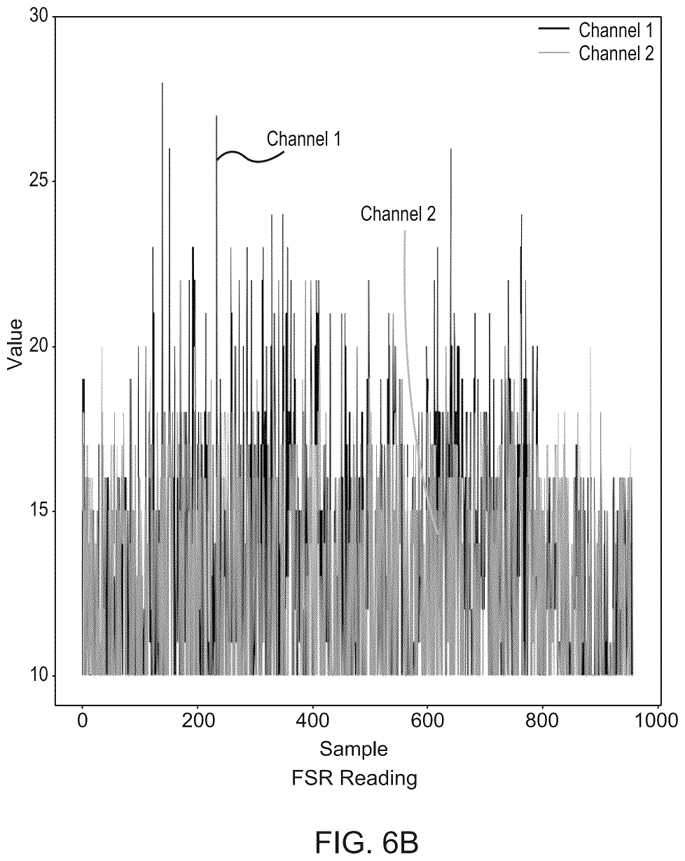

FIGS. 6A and 6B illustrate an example workflow diagram showing how the disclosed system 100 may acquire evaluate the data to assess whether a patient has dysgraphia. At 1000, the electromyography band 400 is attached to a patient's arm and the patient is asked to write on a pad of paper using the writing utensil 200. At 1005, the various sensors (i.e., the force sensors 230 (FSR1, FSR2) and surface electromyography sensors 410 (sEMG)) collect data from the patient. At 1010, the pre-processing module 320 processes the data and converts the data into the full features at 1015. At 1020, the machine learning model analyzes the full features and at 1025 the machine learning model provides a probability of whether the patient has dysgraphia.

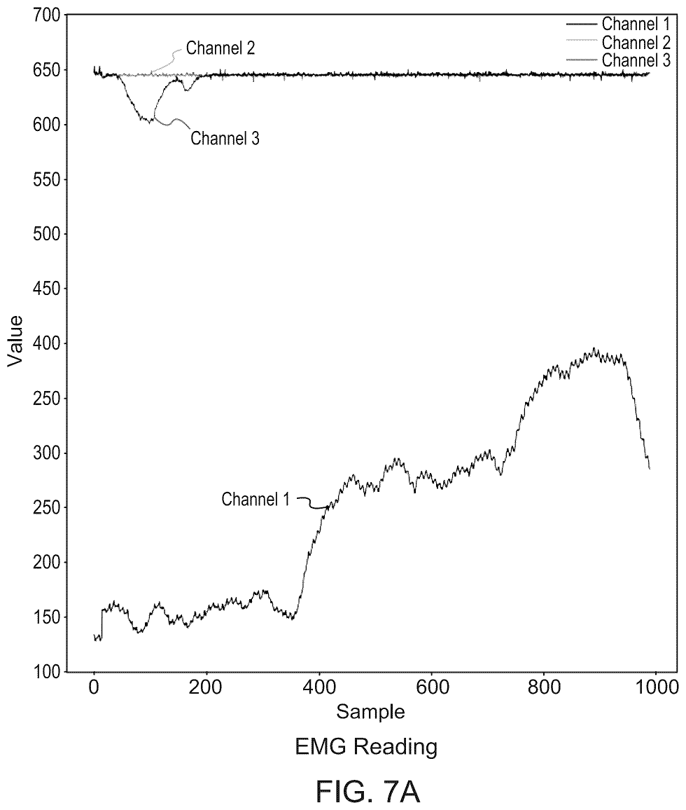

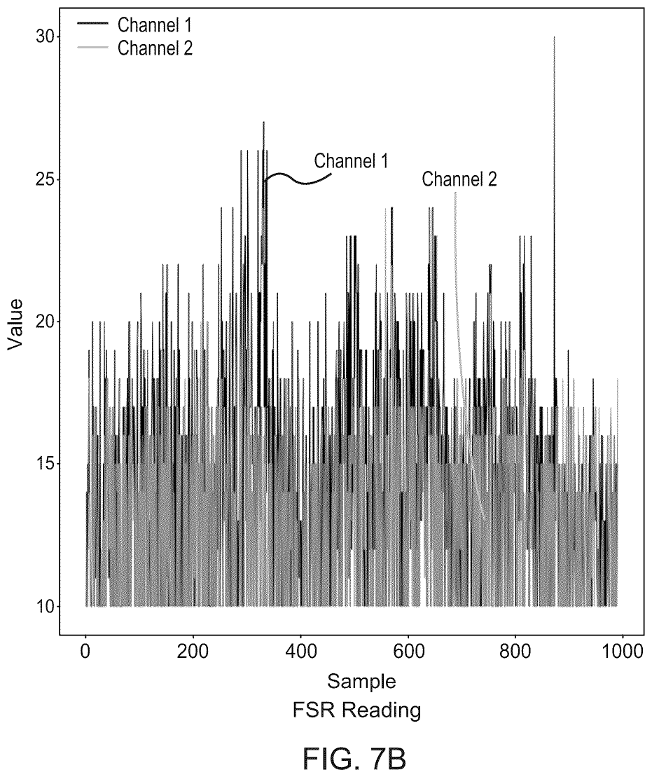

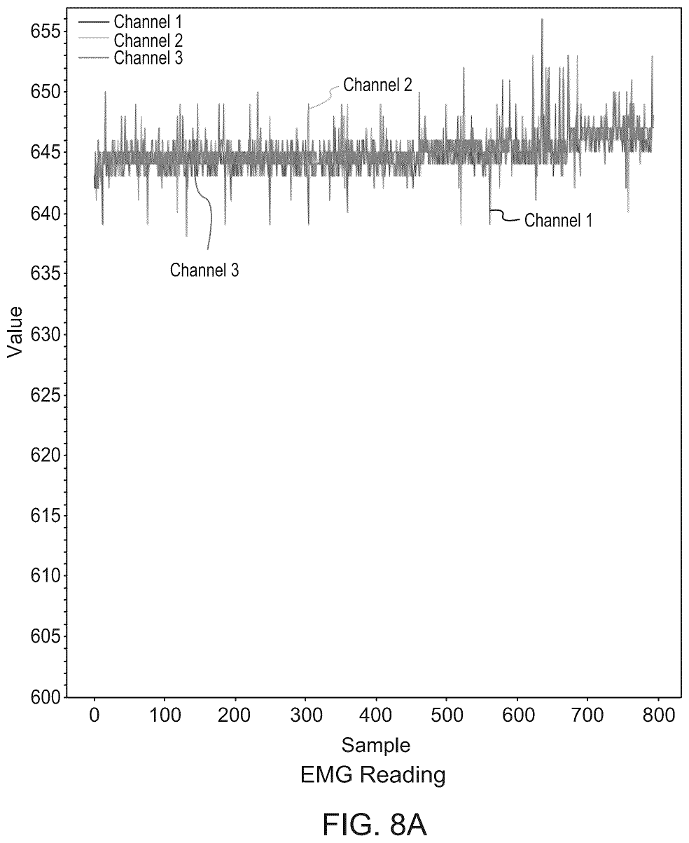

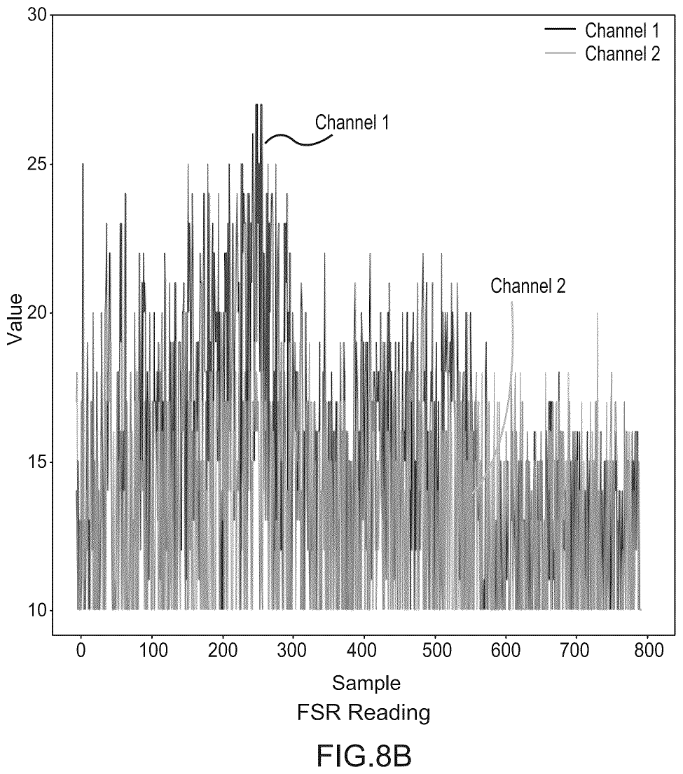

The system's 100 combination of sensors captures both grip pressure and muscle coordination data during the act of writing, offering a multi-dimensional perspective on motor skills. FIGS. 6-8 show example data gathered by the system as a patient executes various writing tasks. FIGS. 6A and 6B show readings from the writing utensil and electromyograph band, respectively, as a patient writes various letters. FIGS. 7A and 7B show readings from the writing utensil and electromyograph band, respectively, as a patient writes a word. FIGS. 8A and 8B show readings from the writing utensil and electromyograph band, respectively, as a patient draws a square.

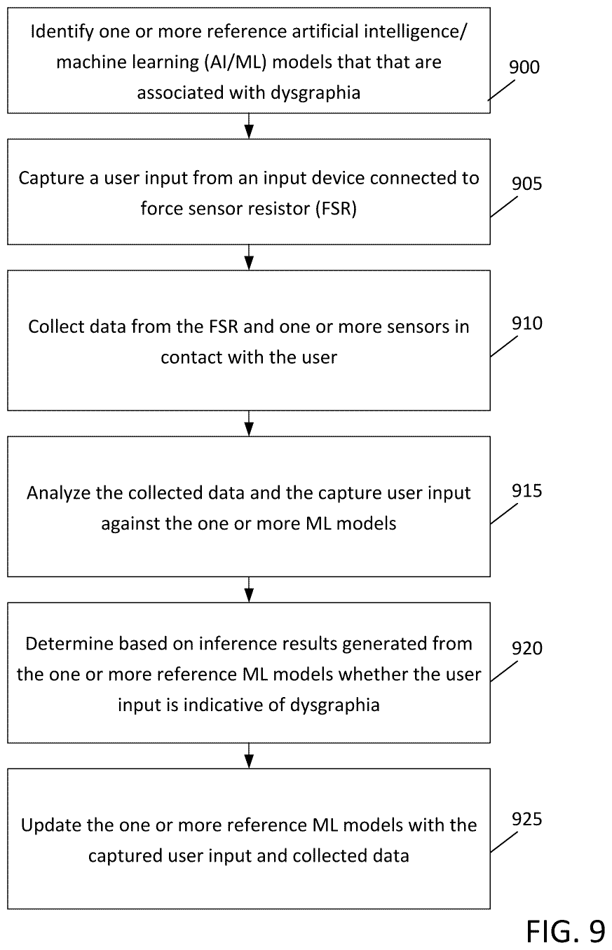

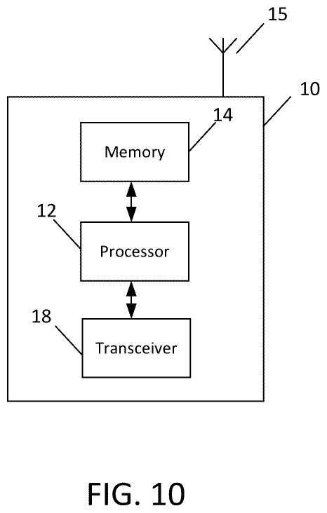

FIG. 9 illustrates an example flow diagram of a method, according to certain embodiments. In an embodiment, the method of FIG. 9 may be performed by elements of the system 100 including, but not limited to, for example, the computer, as illustrated in FIGS. 1 and 10.

According to certain embodiments, the method of FIG. 9 may include, at 900, identifying one or more reference artificial intelligence or machine learning (ML) models that that are associated with dysgraphia. The method may also include, at 905, capturing a user input from an input device connected to force sensor resistor (FSR). The method may further include, at 910, collecting data from the FSR and one or more sensors in contact with the user. In addition, the method may include, at 915, analyzing the collected data and the capture user input against the one or more ML models. The method may also include, at 920, determining based on inference results generated from the one or more reference ML models whether the user input is indicative of dysgraphia. The method may further include, at 925, updating the one or more reference ML models with the captured user input and collected data.

According to certain embodiments, the user input may be the user's handwriting. According to some embodiments, the collected data from the FSR may be the user's grip pressure. According to other example embodiments, the one or more sensors may include electronic sensors for capturing the user's physiological data. According to further embodiments, the user's physiological data captured may be muscle activation data.

In certain embodiments, the is muscle activation data may be captured from muscle movements of the wrist and biceps. In some embodiments, the one or more sensors may be a Surface Electromyography (sEMG) sensor band.

FIG. 10 illustrates an apparatus 10 according to certain example embodiments. In certain example embodiments, apparatus 10 may be an any of the hardware, computer devices, or other similar device described herein. For example, apparatus 10 may include, but not limited to, for example, sensors, controllers, receivers, computing devices, and other similar devices. In some embodiments, apparatus 10 may be in communication (i.e., connected to either via wire or wirelessly) with other similar computer devices forming a network of connected computer devices.

In some example embodiments, apparatus 10 may include one or more processors, one or more computer-readable storage medium (for example, memory, storage, or the like), one or more radio access components (for example, a modem, a transceiver, or the like), and/or a user interface.

As illustrated in the example of FIG. 10 apparatus 10 may include or be coupled to a processor 12 for processing information and executing instructions or operations. Processor 12 may be any type of general or specific purpose processor. In fact, processor 12 may include one or more of general-purpose computers, special purpose computers, microprocessors, digital signal processors (DSPs), field-programmable gate arrays (FPGAs), application-specific integrated circuits (ASICs), and processors based on a multi-core processor architecture, as examples. While a single processor 12 is shown in FIG. 12, multiple processors may be utilized according to other example embodiments. For example, it should be understood that, in certain example embodiments, apparatus 10 may include two or more processors that may form a multiprocessor system (e.g., in this case processor 12 may represent a multiprocessor) that may support multiprocessing. According to certain example embodiments, the multiprocessor system may be tightly coupled or loosely coupled (e.g., to form a computer cluster.

Processor 12 may perform functions associated with the operation of apparatus 10 including, as some examples, precoding of antenna gain/phase parameters, encoding and decoding of individual bits forming a communication message, formatting of information, and overall control of the apparatus 10, including processes illustrated in FIGS. 1-9.

Apparatus 10 may further include or be coupled to a memory 14 (internal or external), which may be coupled to processor 12, for storing information and instructions that may be executed by processor 12. Memory 14 may be one or more memories and of any type suitable to the local application environment, and may be implemented using any suitable volatile or nonvolatile data storage technology such as a semiconductor-based memory device, a magnetic memory device and system, an optical memory device and system, fixed memory, and/or removable memory. For example, memory 14 can be comprised of any combination of random access memory (RAM), read only memory (ROM), static storage such as a magnetic or optical disk, hard disk drive (HDD), or any other type of non-transitory machine or computer readable media. The instructions stored in memory 14 may include program instructions or computer program code that, when executed by processor 12, enable the apparatus 10 to perform tasks as described herein.

In certain example embodiments, apparatus 10 may further include or be coupled to (internal or external) a drive or port that is configured to accept and read an external computer readable storage medium, such as an optical disc, USB drive, flash drive, or any other storage medium. For example, the external computer readable storage medium may store a computer program or software for execution by processor 12 and/or apparatus 10 to perform any of the methods illustrated in FIGS. 1-9.

In some example embodiments, apparatus 10 may also include or be coupled to one or more antennas 15 for receiving a downlink signal and for transmitting via an uplink from apparatus 10. Apparatus 10 may further include a transceiver 18 configured to transmit and receive information. The transceiver 18 may also include a radio interface (e.g., a modem) coupled to the antenna 15. The radio interface may include other components, such as filters, converters signal shaping components, and the like, to process symbols, carried by a downlink or an uplink.

For instance, transceiver 18 may be configured to modulate information on to a carrier waveform for transmission by the antenna(s) 15 and demodulate information received via the antenna(s) 15 for further processing by other elements of apparatus 10. In other example embodiments, transceiver 18 may be capable of transmitting and receiving signals or data directly. Additionally or alternatively, in some example embodiments, apparatus 10 may include an input and/or output device (I/O device). In certain example embodiments, apparatus 10 may further include a user interface, such as a graphical user interface or touchscreen.

In certain example embodiments, memory 14 stores software modules that provide functionality when executed by processor 12. The modules may include, for example, an operating system that provides operating system functionality for apparatus 10. The memory may also store one or more functional modules, such as an application or program, to provide additional functionality for apparatus 10. The components of apparatus 10 may be implemented in hardware, or as any suitable combination of hardware and software.

Software may be used to fuse and synchronize this collected data in real time, creating a comprehensive representation of how grip pressure and muscle movements interact. This synchronized data may be fed into a machine learning model that, as discussed above, has been trained on a diverse dataset containing samples from individuals with and without dysgraphia. The machine learning model learns to recognize specific patterns within the grip pressure and muscle movement data that may indicate dysgraphia-related motor issues. When the system 100 is in use, the machine learning model may continuously analyze the real-time data and compare it against the learned patterns. If deviations from the expected patterns are identified, the system 100 can promptly signal potential dysgraphia-related concerns. This immediate feedback mechanism enables timely intervention strategies and tailored support for people with dysgraphia.

The system 100 disclosed herein can provide real-time analysis of grip pressure and muscle movement during writing. Unlike known tablet-based techniques, which rely on handwriting captured on a tablet's surface, the disclosed system 100 can directly measure the pressure applied by a patient's hand while gripping the writing utensil 200 and can monitor muscle movements in the forearm via the electromyograph band 400. This real-time data may provide a more comprehensive and accurate understanding of the interaction between grip pressure, muscle coordination, and handwriting execution. This approach also offers insights into motor skill patterns that may be indicative of dysgraphia, enabling earlier and more precise diagnosis compared to methods that primarily focus on the visual, dynamic and kinematic aspects of handwriting.

Furthermore, the system 100 disclosed herein can provide significant improvements to the functioning of various technologies and technical fields in the future. For example, by providing real-time analysis of grip pressure and muscle movement during writing, it may be possible to enhance education technology, enabling educators to diagnose dysgraphia accurately and personalize interventions for better learning outcomes. In the realm of medical diagnostics, the disclosed system may empower medical professionals to precisely identify motor skill challenges, leading to tailored therapeutic strategies. Additionally, the disclosed system may advance assistive technology, potentially offering individuals with dysgraphia insights to improve their writing abilities.

Additionally, the integration of a machine learning classifier within the system 100 may showcase the potential of artificial intelligence to understand intricate motor behaviors, while the incorporation of force sensors 230 and electromyography sensors 410 will contribute to the evolution of sensor technology. In this way, certain embodiments hold the promise of reshaping and elevating multiple technical fields for the better.

The system 100 and method disclosed herein provide several advantages known dysgraphia assessments. For instance, certain embodiments may provide a data collection framework (hardware as well as software) along with analysis for dysgraphia diagnosis. Furthermore, certain embodiments may explore the muscle movements of the wrist and biceps using surface electromyography sensors 410 (literature focused on palm muscles movement) and pen-holding grip using force sensors 230. Known dysgraphia assessments do not utilize a design or combination of sensors for extracting the grip pattern as well as muscle coordination as those of certain embodiments described herein.

Additionally, current artificial intelligence-based systems used for dysgraphia diagnosis primarily rely on either analyzing online handwriting data captured by tablet devices or conducting analyses of offline images. These diagnostic methods for dysgraphia lack the necessary granularity to capture intricate motor skill patterns accurately. Moreover, other known methods heavily rely on subjective visual assessments, making them susceptible to human bias. By integrating force sensors 230 for grip pressure and electromyography sensors 410 for muscle movement into a system, objective and quantitative insights into dysgraphia-related motor challenges can be provided. Thus, the system 100 and method disclosed herein not only address the limitations of existing dysgraphia assessments, but also pave the way for a more accurate and immediate diagnostic tool.

In another embodiment, the certain example embodiments discussed above may be implemented with a deep neural network (DNN). The DNN may include an input layer, a plurality of hidden layers, and an output layer. It is understood that alternative embodiments may have any number of two or more hidden layers. Each may have one or more nodes. Other neural network structures are also possible in alternative embodiments of the DNN, in which not every node in each layer is connected to every node in the previous and next layers. Each node in the input layer can be assigned a value and output that value to every node in the next layer (e.g., hidden layer). The nodes in the input layer can represent features about a particular environment or setting. Each node in the hidden layers can receive an outputted value from nodes in a previous layer (e.g., input layer) and associate each of the nodes in the previous layer with a weight. Each hidden node can then multiply each of the received values from the nodes in the previous layer with the weight associated with the nodes in the previous layer and output the sum of the products to each node in the next layer. Nodes in the output layer handle input values received from the nodes in the hidden layer in a similar fashion. In one example, each output node in the output layer may multiply each input value received from each node in the previous layer (e.g., hidden layer) with a weight and sum the products to generate an output value. The output value of each output node can output information in a predefined format, where the information has some relationship to the corresponding information from the previous layer. Example outputs may include, but are not limited to, classifications, relationships, measurements, instructions, and recommendations. For example, a DNN that classifies whether the captured data from the invention corresponds to reference instances of dysgraphia. The output nodes can also be used to classify any of a wide variety of objects and other features and otherwise output any of a wide variety of desired information in desired formats.

In another embodiment, the DNN for the detecting dysgraphia may be trained using the following method for training DNNs i. The method comprises the following steps: feeding an input to the DNN; generating one or more task related predictions of the input; concurrently estimating a prediction uncertainty of the one or more task-related predictions; and determining whether to accept the one or more task related predictions based on the prediction uncertainty. In another embodiment, to begin training the deep neural network (DNN), initial weights may be chosen randomly or by pre-training using a deep belief network. The training cycle can then be performed in either a supervised or unsupervised manner.

Supervised learning uses a training set to teach models to yield the desired output. The training dataset includes inputs and desired outputs, which allow the model to learn over time, or when the training dataset includes input having known output and the output of the neural network is manually graded. The network processes the inputs and compares the resulting outputs against a set of expected or desired outputs. Errors are then propagated back through the system. The training framework can adjust to change the weights that control the untrained neural network. The training framework can provide tools to monitor how well the untrained neural network is converging towards a model suitable for generating correct answers based on known input data. The training process repeatedly occurs as the network weights are adjusted to refine the output generated by the neural network. The training process can continue until the neural network reaches a statistically desired accuracy associated with a trained neural network. The trained neural network can then be deployed to implement any number of machine learning operations to output a result.

Supervised learning is typically separated into two types of problems-classification and regression. Classification uses an algorithm to assign test data accurately into specific categories. Regression is used to understand the relationship between dependent and independent variables. Numerous different algorithms and computation techniques can be used in supervised machine learning, including but not limited to, neural networks, naïve bayes, linear regression, logistic regression, support vector machines (SVM), k-nearest neighbor, and random forest.

Unsupervised learning is a learning method in which the network uses algorithms to analyze and cluster unlabeled data. These algorithms discover hidden patterns or data groupings. Therefore, the training dataset includes input data without any associated output data. The untrained neural network can learn groupings within the unlabeled input and determine how individual inputs relate to the overall dataset. Unsupervised training can be used to for three main tasks-clustering, association, and dimensionality. Clustering is a data mining technique that groups unlabeled data based on similarities and differences. This technique is often used to process raw, unclassified data objects into groups represented by structures or patterns in the information. Association is a rule-based method for finding relationships between variables in a given dataset. This method is often used for market basket analysis. Dimensionality reduction is used when a given dataset's number of features (dimensions) is too high. This technique is commonly used in the preprocessing of data.

Variations of supervised and unsupervised training may also be employed. Semi-supervised learning is a technique in which the training dataset includes a mix of labeled and unlabeled data of the same distribution. Incremental learning is a variant of supervised learning in which input data is continuously used to train the model further. Incremental learning enables the trained neural network to adapt to the new data without forgetting the knowledge instilled within the network during initial training.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

One having ordinary skill in the art will readily understand that the invention as discussed above may be practiced with steps in a different order, and/or with hardware elements in configurations which are different than those which are disclosed. Therefore, although the invention has been described based upon these example embodiments, it would be apparent to those of skill in the art that certain modifications, variations, and alternative constructions would be apparent, while remaining within the spirit and scope of example embodiments.

We claim:

1. A computer implemented method for diagnosing dysgraphia, comprising:
identifying one or more reference machine learning (ML) models that that are associated with dysgraphia;
capturing input from a user via an input device connected to a force sensor resistor (FSR);
collecting data from the FSR and one or more sensors in contact with the user;
analyzing the collected data and the capture user input against the one or more reference ML models;
determining based on inference results generated from the one or more reference ML models whether the user input is indicative of dysgraphia; and
updating the one or more reference ML models with the captured input and collected data.

2. The computer implemented method of claim 1, the input is the user's handwriting.

3. The computer implemented method of claim 1, wherein the collected data from the FSR is user's grip pressure.

4. The computer implemented method of claim 1, wherein the one or more sensors comprise electronic sensors for capturing the user's physiological data.

5. The computer implemented method of claim 4, wherein the user's physiological data captured is muscle activation data.

6. The computer implemented method of claim 5, wherein the is muscle activation data is captured from muscle movements of the user's wrist and biceps.

7. The computer implemented method of claim 1, wherein the one or more sensors is a Surface Electromyography (sEMG) sensor band.

8. A device for diagnosing dysgraphia, comprising:
at least one processor; and
at least one memory storing instructions, wherein the instructions, when executed by the at least one processor, cause the apparatus at least to:
identify one or more reference machine learning (ML) models that that are associated with dysgraphia;
capture input from a user from an input device connected to force sensor resistor (FSR);
collect data from the FSR and one or more sensors in contact with the user;
analyze the collected data and the captured input against the one or more reference ML models;
determine based on inference results generated from the one or more reference ML models whether the input is indicative of dysgraphia; and
update the one or more ML models with the captured input and collected data.

9. The device of claim 8, wherein the instructions, when executed by the at least one processor, further cause the device to:
analyze the input is the user's handwriting.

10. The device of claim 8, wherein the collected data from the FSR is the user's grip pressure.

11. The device of claim 8, wherein the one or more sensors comprise electronic sensors for capturing the user's physiological data.

12. The device of claim 11, wherein the user's physiological data captured is muscle activation data.

13. The device of claim 12, wherein the muscle activation data is captured from muscle movements of the user's wrist and biceps.

14. The device of claim 8, wherein the one or more sensors is a Surface Electromyography (sEMG) sensor band.

15. The device of claim 8, wherein the instructions, when executed by the at least one processor, further cause the device to:
train the one or more reference ML models with the captured input and collected data along with additional captured input and additional collected data.

16. The device of claim 15, wherein training of the one or more reference ML models is based on at least one of a specific group to which the user belongs, and a condition associated with the user.
